(19) United States
(12) Patent Application Publication
(10) Pub. No.: US 2003/0065373 A1
(43) Pub. Date: Apr. 3, 2003

(54) Medical device having rheometric materials and method therefor

(76) Inventors: Eric G. Lovett, Roseville, MN (US); Robert J. Sweeney, Woodbury, MN (US); David T. Jacobsen, San Jose, CA (US); Gildo L. Epis JR., San Jose, CA (US)

Correspondence Address: Schwegman, Lundberg, Woessner & Kluth, P.A., P.O. Box 2938, Minneapolis, MN, 55402, US

(21) Appl. No.: 09/970,146

(22) Filed: Oct. 2, 2001

(51) Int. Cl. A61N 1/05

(52) U.S. Cl. 607/122

(57) ABSTRACT

A medical device includes a device body which extends from a proximal end to a distal end. The medical device also includes a rheometric material associated therewith. Examples of rheometric materials include, but are not limited to, electroactive materials, such as a polymer or magnoactive material. The rheometric material stiffens at least a portion of the device body as electric current, voltage, or a magnetic field is applied thereto.

### TECHNICAL FIELD

The present invention relates generally to medical devices. More particularly, it pertains to medical devices which include rheometric materials associated therewith.

### BACKGROUND

Medical devices such as leads are implanted in or about the heart have been used to reverse certain life threatening arrhythmias, or to stimulate contraction of the heart. Electrical energy is applied to the heart via the leads to return the heart to normal rhythm. Leads have also been used to sense in the atrium or ventricle of the heart and to deliver pacing pulses to the atrium or ventricle. Technically, the pacemaker or the automatic implantable cardioverter defibrillator receives signals from the lead and interprets them. The same lead used to sense the condition is sometimes also used in the process of delivering a corrective pulse or signal from the pulse generator of the pacemaker.

Cardiac pacing may be performed by the transvenous method or by leads implanted directly onto the ventricular epicardium. Most commonly, permanent transvenous pacing is performed using a lead positioned within one or more chambers of the heart. The lead may also be positioned in both chambers, depending on the lead, as when a lead passes through the atrium to the ventricle. Electrodes of the lead may be positioned within the atrium or the ventricle of the heart. For other applications, the lead may be positioned in cardiac veins or arteries, for example, through use of a guide catheter. Depending on the application, the precise location of the lead relative to the heart can be critical. To position a lead or other medical devices, a stiff guidewire is used. Alternatively, a stiff stylet is disposed within the medical device and is guided to maneuver the medical device. However, each of the devices stiffen the entire lead or medical device, which does not provide for positioning the medical device within difficult to reach locations, such as complex vasculature near the heart. In another approach, a physician will manually apply torque to the medical device to position, maneuver, or maintain positioning of the medical device. However, this may result in discomfort to the physician and/or present a distraction during the procedure.

Positioning an electrode disposed on a distal end of a medical device within a vein or artery presents additional challenges in maintaining the lead in a fixed position since the distal end of the lead does not abut a surface. In addition, positioning a device near contracting tissue, such as a beating heart provides additional challenges in positioning and/or bracing a medical device in a specific position since the body moves and/or repeatedly is moving. Furthermore, body mechanics such as blood flow or blood pressure provides a challenging environment in which to maneuver a medical device. These challenges also may result in poor results from the medical device, for example the pacing, sensing, or shocking capabilities of a lead can be affected from poor placement within a patient. If a device is not properly placed, this may further lead to a shortened device life.

Therefore, what is needed is a medical device which can be positioned within complex locations of a patient, and can be placed under rigorous conditions. There is also a need for a medical device, such as a lead or a guide catheter, which can be accurately maneuvered and placed in, on, or near a beating heart of a patient or within complex vasculature of a patient.

### SUMMARY

A medical device includes a device body with rheometric material associated therewith. The device body extends from a proximal end to a distal end. One or more electrodes are coupled with the device body, where the electrode is configured to transmit and receive electrical signals to and from tissue, and a rheometric material is electrically coupled with the electrodes. The rheometric material optionally comprises a layer of material disposed on an outer surface of the electrode. The rheometric material includes, but is not limited to, an electroactive polymer or magnoactive material, as further discussed below.

Several options for the medical device are as follows. For instance, in one option, the rheometric material comprises a coating of electroactive polymer having a thickness of about 180 micron. In another option, the rheometric material comprises a strip of material wound around a longitudinal axis of the device body. Other options include disposing the assembly on the first surface of the device and/or a second surface of the device body, where the first surface is optionally opposite the second surface.

In another embodiment, a medical device comprises an elongate device body extending from a proximal end to a distal end, an at least one assembly coupled with the device body, where the at least one assembly is configured to stiffen the device body. The device further includes a rheometric material, such as an electroactive polymer, where the rheometric material contracts and/or stiffens when current is applied thereto.

Several options for the medical device are as follows. For instance, in one option, the medical device further includes a control system which selectively applies current to the rheometric material, and a means for providing feedback to the control system. In another option, the medical device further includes a means for transferring fluid along the elongate device body. Alternatively, the device body includes a plurality of assemblies, and the device body has a generally circular cross-section or a generally square cross-section. In yet another option, the medical device further includes a means for selectively stiffening intermediate portions of the device body. Still further, the medical device includes the options discussed above.

In another embodiment, a medical device includes a device body extending from a proximal end to a distal end, at least one assembly coupled with the device body, the at least one assembly comprises a winding of material wound around a longitudinal axis of the device body, where the at least one assembly is configured to stiffen the device body. The assembly includes a rheometric material, where the rheometric material contracts and/or stiffens when current is applied thereto. The rheometric material includes, but is not limited to, an electroactive polymer and/or magnoactive material.

In yet another embodiment, a medical device includes an elongate device body extending from a proximal end to a distal end, at least one assembly coupled with the device body, and a means for electrically stiffening the at least one assembly and the device body. Several options for the device are as follows. For instance, in one option, the assembly includes an electroactive polymer or a magnoactive material associated therewith. In another option, the device body includes at least one lumen therein, and rheometric material is disposed within one or more lumens.

In another embodiment, a medical device includes an elongate device body extending from a proximal end to a distal end, for instance, a guide catheter. The device body includes at least one lumen therein, and rheometric material is disposed within one or more lumens. The rheometric material, such as an electroactive polymer or magnoactive material, is configured to stiffen the elongate device body upon application of electrical energy to the rheometric material. Optionally, the device body includes a passage extending from the proximal end to the distal end, the passage sized to receive at least one instrument therein, and a plurality of lumens are disposed about the passage, one or more lumens filled with rheometric material.

In another embodiment, a method for manipulating a medical device is described herein. It should be noted that the method includes the above and below discussed device embodiments described herein. Although some of the embodiments are discussed in the context of a lead or a catheter, the method applies to a wide variety of medical devices, including, but not limited to, medical devices for chronic or acute use, catheters, leads, endoscopes, ablation tools, pressure measuring tools, or blood sampling devices.

The method includes associating rheometric material with a device body, such as an elongate device body. For instance, the method includes associating at least one assembly with the device body, where the at least one assembly optionally includes at least one electrode. The method further includes applying energy to the rheometric material, stiffening at least a portion of the device body, and manipulating the device body.

Several options for the method are as follows. For instance, in one option, applying energy to the assembly comprises applying voltage to multiple assemblies each including at least one electrode electrically coupled with a layer of electroactive polymer. The energy is optionally applied to each assembly simultaneously, or selectively applied energy to each assembly at different times. Still further, in another option, applying energy includes applying voltage to an assembly which is wound around an axis of the device body, or to an assembly disposed at a distal end of the device body, or to a plurality of assemblies disposed on a single side of the device body, or to a plurality of assemblies disposed on at least two sides of the device body. Optionally, the assemblies are disposed within the device body or are disposed on one or more outer surfaces of the device body.

In other options for the method, the method further includes selectively varying stiffness of the device body, where selectively varying the stiffness of the device body includes moving the device body within a passage, or bracing the device body against movement, or moving fluid through the device body.

In another embodiment, a method includes providing an elongate device body having a length, associating a rheometric material along at least a portion of the length, applying an electric current to the rheometric material, and stiffening at least a first portion of the device body.

Several options for the method are as follows. For instance, in one option, applying electric current includes pulsing the electric current and alternately stiffening and relaxing the first portion of the device body. Alternatively, stiffening includes stiffening the entire length of the device body. In yet another option, the device body includes one or more lumens therein, and associating includes disposing rheometric material in at least one lumen of the device body, or in at least two or more lumens of the device body. In yet another option, applying electric current includes pulsing the electric current and alternately stiffening and relaxing multiple portions of the device body. In yet another option, the device body is preformed with a curve.

The medical device described herein is controllable from an outside source, without having to implement invasive procedures, or without having to rely exclusively on additional instruments such as stylets. In addition, the stiffness of the medical device can also be modified at different portions and at different time periods which allows for the resistance of movement of the device in response to, for example, a beating heart. Alternatively, the ability to selectively and independently modify the stiffness of the lead along different segments, at different times allows for the position of the medical device to be manipulated within the patient, without further invasive procedures, and allows for the device to be manipulated into complex configurations, such as within the human vasculature. Another provided benefit is that the device can be braced against moving tissue, and/or for procedures in which the device moves during the procedure.

Furthermore, since the medical device can be manipulated into more precise locations, under more demanding conditions, improved positioning of the device can be achieved, resulting in improved performance of the medical device. For example, delivering energy to a more favorable location on the heart results in a better chance for a more-effective defibrillation.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 is an elevational view illustrating a medical device constructed in accordance with one embodiment.

FIG. 2 is a cross-sectional view illustrating an electrode assembly constructed in accordance with the one embodiment.

FIG. 3 is an elevational view illustrating a portion of the medical device constructed in accordance with one embodiment.

FIG. 4 is an elevational view illustrating a portion of the medical device constructed in accordance with one embodiment.

FIG. 5 is an elevational view illustrating a portion of the medical device constructed in accordance with one embodiment.

FIG. 6 is an elevational view illustrating a portion of the medical device constructed in accordance with one embodiment.

FIG. 7 is an elevational view illustrating a portion of the medical device constructed in accordance with one embodiment.

FIG. 8 is a cross-sectional view illustrating a portion of the medical device constructed in accordance with one embodiment.

FIG. 9 is a cross-sectional view illustrating a portion of the medical device constructed in accordance with one embodiment.

FIG. 10 is a block diagram illustrating a system of an assembly constructed in accordance with one embodiment.

FIG. 11 is a side elevational view illustrating a medical device constructed in accordance with one embodiment.

FIG. 12 is a cross-sectional view illustrating a portion of the medical device constructed in accordance with one embodiment.

FIG. 13 is a cross-sectional view illustrating a portion of the medical device constructed in accordance with one embodiment.

FIG. 14 is a cross-sectional view illustrating a portion of the medical device constructed in accordance with one embodiment.

FIG. 15 is a cross-sectional view illustrating a portion of the medical device constructed in accordance with one embodiment.

FIG. 16 is a perspective view illustrating a portion of the medical device constructed in accordance with one embodiment.

FIG. 17 is a perspective view illustrating a portion of the medical device constructed in accordance with one embodiment.

FIG. 18 is a perspective view illustrating a portion of the medical device constructed in accordance with one embodiment.

FIG. 19 is a perspective view illustrating a portion of the medical device constructed in accordance with one embodiment.

FIG. 20 is a side view illustrating a portion of the medical device constructed in accordance with one embodiment.

FIG. 21 is a cross-section view taken along A-A of FIG. 20 illustrating a portion of the medical device constructed in accordance with one embodiment.

FIG. 22 is a block diagram illustrating a method in accordance with one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

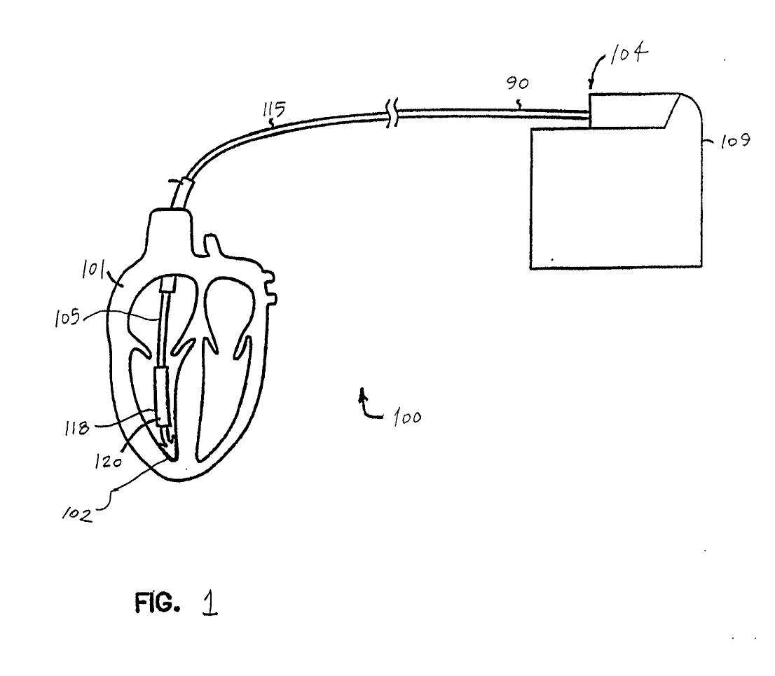

FIG. 1 illustrates a medical device 90, for example, an elongate medical device, including rheometric material associated therewith. When electric current is applied to the rheometric material, the rheometric material causes the medical device 90 to stiffen. The rheometric material includes, but is not limited to, solids and liquids, and electroactive or magnoactive materials, as further described below. Examples of the medical device 90 include, but are not limited to: medical device for chronic or acute use, catheter, lead, endoscope, ablation tool, pressure measuring tool, endoscope, or a blood sampling device. The medical device 90 can be placed in a variety of locations within a patient. In one option, the medical device 90 comprises a single-pass lead 100 for delivering electrical pulses to stimulate a heart 101 and/or for receiving electrical pulses to monitor the heart 101. Although the device 90 is illustrated in one example as a lead placed within a heart, it is not strictly limited to the lead 100 and is not limited to placement within the heart.

The lead 100 extends from a distal end 102 to a proximal end 104, and has an intermediate portion 105 therebetween. The distal end 102 is adapted for implantation within the heart 101 of a patient. The proximal end 104 of the lead 100 has a terminal connector which electrically connects the various electrodes and conductors within the lead body to a pulse generator and signal sensor 109. Although shown disposed within the right ventricle of the heart 101, the medical device 90 is also suitable for use in other parts of a patient, for instance, within a vein, artery, or other locations. The pulse generator and signal sensor 109 contains electronics to sense various electrical signals of the heart and also produce current pulses for delivery to the heart 101.

The lead 100 includes a lead body 115, an elongate conductor contained within the lead body 115, and optionally at least one electrode assembly 120 having at least one electrode 118 coupled with the elongate conductor. Optionally, the elongate conductor comprises a coiled conductor and defines a lumen therein and thereby is adapted to receive an optional stylet that extends through the length of the lead 100. The lead body 115 includes a biocompatible insulating material and forms an outer surface of the lead 100.

Optionally, the stylet is used to further stiffen and/or maneuver the lead 100, and is manipulated to facilitate the insertion of the lead 100 into and through a vein and through an intracardiac valve to advance the distal end 102 of the lead 100 into, for example, the ventricle of the heart 101 . A stylet knob is coupled with the stylet for rotating the stylet, advancing the conductor into tissue of the heart, and for manipulating the lead 100. Alternatively, the elongate conductor comprises a cable conductor. It should be noted that the stylet can optionally be used in conjunction with the various medical devices 90 discussed above and below, although the stylet is not required.

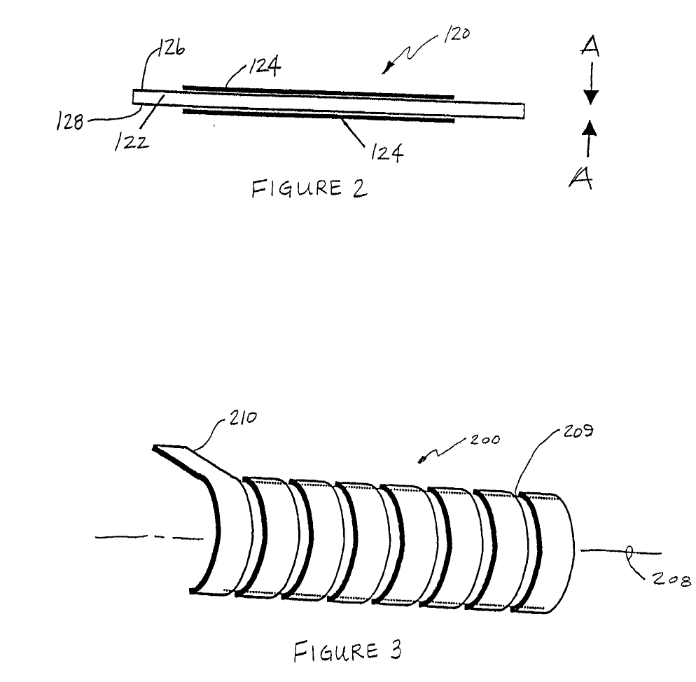

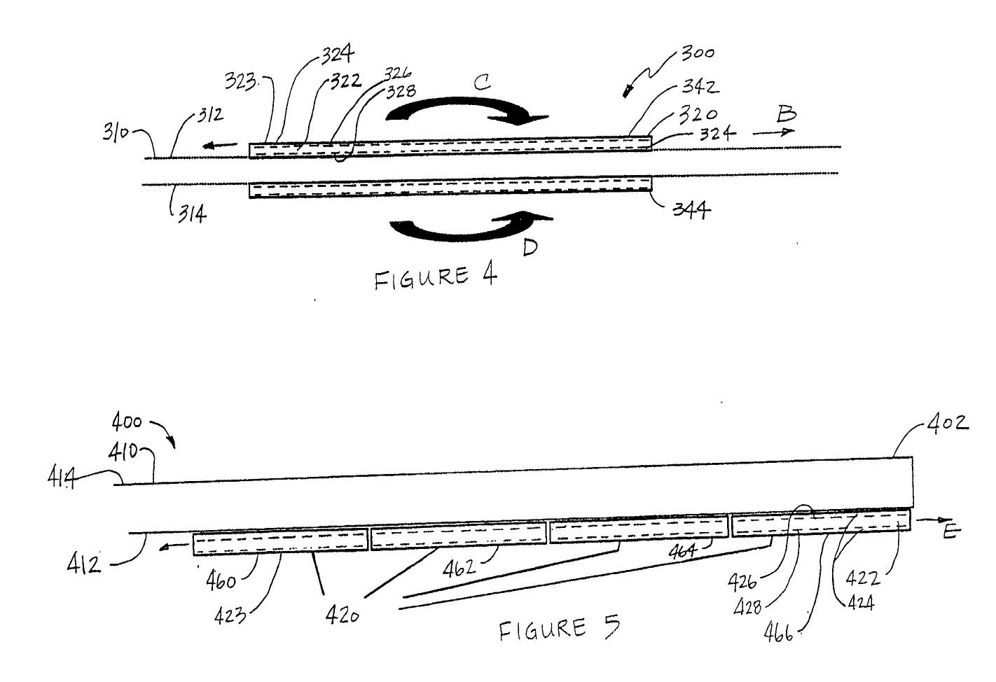

FIG. 2 illustrates one example of an electrode assembly 120. It should be noted that the at least one electrode assembly 120 can be used primarily to stiffen the device body. Alternatively, the at least one electrode assembly 120 can be used to both stiffen the device body, as further described below, and used as a sensing, pacing, or defibrillation electrode, or an electrode which electrically stimulates or monitors tissue. In yet another option, at least one electrode assembly 120 is used to stiffen the device, at least another electrode assembly 120 is used as a stimulating or sensing electrode, and the electrode assemblies are electrically coupled together.

The electrode assembly 120 includes rheometric material associated therewith. For instance, the electrode assembly 120 includes a layer of an electrically active polymer 122 with electrodes 124 deposited thereon. Examples of suitable electrically active polymers include, but are not limited to, nation, flemion, ionic polymer metallic composite (IPMC), and ionic polymers such as polypyrole, polyethylenedroxythrophene, polyaniline, poly-(p-phenylene vinylene)s, polythiophenes. In one example, the layer of electrically active polymer 122 is a film of polymer about 180 micron thick. Other thicknesses of the layer of electrically active polymer 122 are suitable as well. For instance, a thickness of 0.2 mm of nafion is one example. In another example, a layer of less than 50 μm is suitable. In another option, the electrode assembly 120 includes rheometric materials associated therewith. Rheometric materials experience a stiffness change when small amounts of current or magnetic field are applied, and the material undergoes a phase change. Examples of rheometric materials include, but are not limited to, electrorheological materials, such as polyvinyl chloride nonionic gel with dioctyl phthalate. Other examples of rheometric materials include magneto-rheological fluids, which have, for instance, an oil base, water base, or silicone base. Such magneto-rheological materials can be obtained from the Lord Corporation of North Carolina.

The electrodes 124, in one option, comprise a metallic coating which is deposited on opposite surfaces 126, 128 of the layer of electrically active polymer 122. It should be noted that the electrodes 124 are planar or non-planar. In one example, the metallic coating is chemically deposited on the opposite surfaces 126, 128. In another example, the metallic coating is comprised of platinum. Other examples of suitable material include, but are not limited to, gold. The electrodes 124 allow for a voltage to be applied across the layer of electrically active polymer 122. When voltage is applied to the electrodes, for example 2-7 volts, an electric field is established, which causes the layer of electrically active polymer 122 to contract in the direction noted as “A.” As the layer of electrically active polymer 122 contracts along “A”, the electrode 120 stiffens. It should be noted that any rheometric material which causes the electrode 120 to stiffen or contract is suitable for use with the electrode 120.

FIG. 3 illustrates another example of a medical device such as a portion of a lead 200. For instance, the lead 200 is defined in part by a lead body 209 and a longitudinal axis 208. One or more strips of material 210 are disposed along the lead 200. In one option, the one or more strips of material 210 are wound around the lead body 209 and around the axis 208 of the lead 200. It should be noted that the one or more strips of material 210 optionally extends the full length of the lead 200. Alternatively, the one or more strips of material 210 are disposed on portions of the lead 200, or multiple portions of the lead 200, on an outer surface of the body 209, or within the lead body 209.

The one or more strips of material 210 are optionally one continuous strip of material, and are comprised of a rheometric material, for example, any of the rheometric materials discussed above. In one option, the material comprises a layer of polymer 122 with electrodes 124 deposited thereon, as shown in FIGS. 1 and 2. The layer of polymer 122 comprises an electrically active polymer. In one example, the layer of electrically active polymer 122 is a film of polymer about 180 micron thick. Other thicknesses of the layer of electrically active polymer 122 are suitable as well. The electrodes 124, which are electrically coupled with a conductor of the lead 200 (FIG. 3), comprise a metallic coating which is deposited on opposite surfaces 126, 128 of the layer of electrically active polymer 122. In one example, the metallic coating is comprised of platinum. The passive properties of the layer of electrically active polymer 122 and the metallic coating are modifiable to alter the flexibility of the lead 200 (FIG. 3). It should be noted that the electrodes 124 optionally operate to stimulate tissue. For example, the electrodes 124 electrically couple the one or more strips of material 210 (FIG. 3) with an energy source.

The electrodes 124 allow for a voltage to be applied across the layer of electrically active polymer 122. When voltage is applied to the electrodes, for example 2-7 volts, an electric field is established, which causes the layer of electrically active polymer 122 to contract. As the layer of electrically active polymer 122 contracts, the layer of electrically active polymer 122 is forced to expand in the axial and transverse directions. The axial expansion causes the lead body to become stiff as the compressive forces between the windings of the electrically active polymer 122 are increased.

FIG. 4 illustrates yet another embodiment of a medical device, such as a lead 300, where the lead 300 has a lead body 310, including a first surface 312 and a second surface 314 opposite the first surface 312. It should be noted that, although the embodiment is discussed in the context of a lead, this, as well as above and below discussed embodiments, can be incorporated into other medical devices, such as those discussed above. One or more assemblies 320 are coupled with the lead body 310, as further discussed below. Optionally, the one or more assemblies 320 comprises a first assembly 342 and a second assembly 344, where the first assembly 342 is coupled with the first surface 312 and the second assembly 344 is coupled with the second surface 314. The one or more assemblies 320 also optionally comprise an electrode 323 and are adapted to provide and/or receive electrical signals to and from a heart. The electrode 323 is electrically coupled with a conductor of the lead 300.

The first assembly 342 and the second assembly 344 each include a layer of rheometric material. For example, the first assembly 342 and the second assembly 344 include a layer of electroactive polymer 322 with electrodes 324 deposited thereon. In another option, the first assembly 342 and/or the second assembly 344 include a rheometric material, such as magnoactive material or an electroactive polymer without electrodes 324 thereon. In one example, the layer of electrically active polymer 322 is a film of polymer about 180 micron thick. Other thicknesses of the layer of electrically active polymer 322 are suitable as well. The electrodes 324 comprise a metallic coating which is deposited on opposite surfaces 326, 328 of the layer of electrically active polymer 322.

In one example, the metallic coating is comprised of platinum. The electrodes 324 allow for a voltage to be applied across the layer of electrically active polymer 322. However, energy can be supplied to the rheometric material in other methods, such as conductors, as further discussed below. When voltage is applied to the electrodes, for example 2-7 volts, an electric field is established, which causes the layer of electrically active polymer 322 to contract. As the layer of electrically active polymer 322 contracts in a first direction, it also expands along “B,” causing the lead body 310 to bend toward the opposite side in a bending moment. However, an assembly disposed on the opposite side would prevent the lead body 310 from bending, and when undergoing the same type of bending moment.

For example, as voltage is applied to the first assembly 342, the electrically active polymer 322 of the first assembly 342 expands along “B” and produces a bending moment “C” to the lead body 310. As voltage is applied to the second assembly 344, for example, at the same time voltage is applied to the first assembly 342, the electrically active polymer 322 of the second assembly 344 expands along “B” and produces a bending moment “D” to the lead body 310. When the bending moment “C” is opposite the bending moment “D,” the lead body 310 is stiffened by the opposing bending moments.

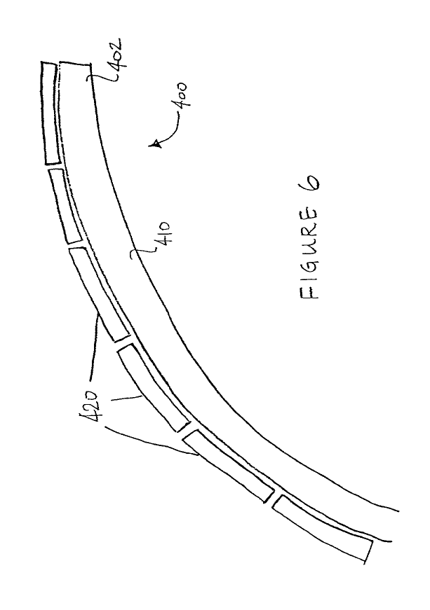

FIG. 5 illustrates yet another alternative of a medical device, such as a lead 400. The lead 400 includes a lead body 410 having a first surface 412 which is opposite a second surface 414. The lead body 410 extends to a distal end 402. One or more assemblies 420 are coupled with the lead body 410 on the first surface 412. The one or more assemblies 420 are serially disposed along the lead body 410, and allow for the assemblies to be selectively activated. For example, a first assembly 460 is disposed along the lead body 410, a second assembly 462 is disposed adjacent to the first assembly 460, a third assembly 464 is disposed adjacent to the second assembly 462, and a fourth assembly 466 is disposed adjacent to the third assembly 464. Optionally, the fourth assembly 466 is disposed at or near the distal end 402 of the lead body 410. The one or more assemblies 420 also optionally comprise an electrode 423 and are adapted to provide and/or receive electrical signals to and from a heart. Each electrode 423 is electrically coupled with a conductor of the lead 400.

The one or more assemblies 420 include rheometric material therewith, where the rheometric material stiffens the body upon application of energy thereto. The rheometric material includes, but is not limited to, magnoactive material or an electroactive polymer. For instance, the one or more assemblies 420 each comprise a layer of polymer 422 with electrodes 424 deposited thereon. The layer of polymer 422 comprises an electrically active polymer. In one example, the layer of electrically active polymer 422 is a film of polymer about 180 micron thick. Other thicknesses of the layer of electrically active polymer 422 are suitable as well. The electrodes 424, which are electrically coupled with a conductor of the lead 400, comprise a metallic coating which is deposited on opposite surfaces 426, 428 of the layer of electrically active polymer 422. In one example, the metallic coating is comprised of platinum. The passive properties of the layer of electrically active polymer 422 and the metallic coating are modifiable, as well as the serial placement of the one or more assemblies 420, to alter the flexibility of the lead 400.

The electrodes 424 allow for a voltage to be applied across the layer of electrically active polymer 422. When voltage is applied to the electrodes, for example 2-7 volts, an electric field is established, which causes the layer of electrically active polymer 422 to contract. As the layer of electrically active polymer 422 contracts in a first direction, it also expands along “E,” causing the lead body 410 to bend toward the opposite side in a bending moment. Having multiple assemblies 420 disposed along the lead 400 allows for the lead 400 to bend.

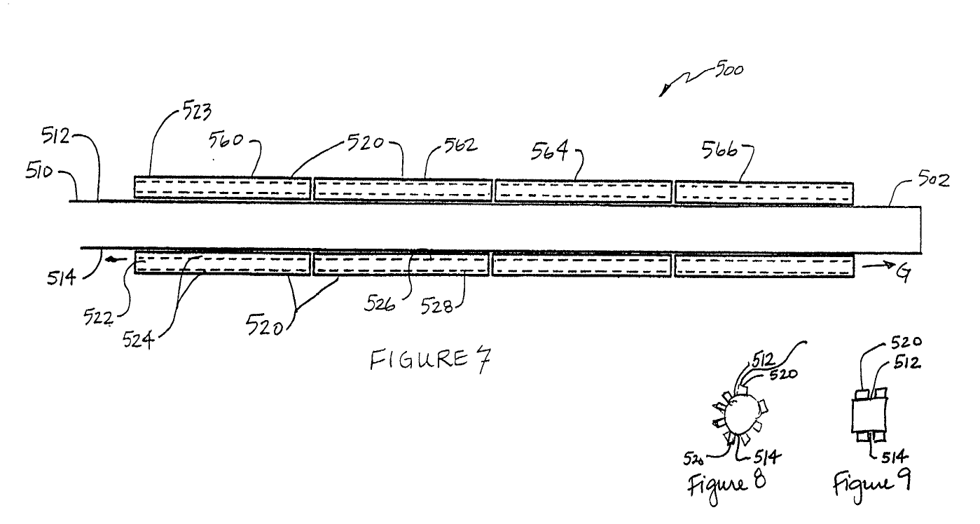

For example, as voltage is applied to the fourth assembly 466, the electrically active polymer 422 of the fourth assembly 466 expands along “E” and produces a bending moment to the distal end 402 of the lead body 410. As voltage is applied to the any of the first, second, and third assemblies 460, 462, 464, for example, at the same time voltage is applied to the first assembly 442, the electrically active polymer 422 of the second assembly 444 expands along “E” and produces an even greater bending moment to the lead body 410, and forces the lead body 410 to curve, as shown in FIG. 6. Since the various assemblies 460, 462, 464, 466 can have voltage selectively applied thereto, bending moments can be applied to various portions of the lead body 410. For instance, applying voltage to the fourth assembly 466 would allow for only the distal end 402 of the lead body 410 to undergo a bending moment, and only the distal end 402 of the lead body 410 would curve, thereby providing the ability to remotely steer the distal end 402 of the lead body 410. Alternatively, the voltage is applied to the various assemblies or segments independently and not at the same time. The result is that the bending of the lead body 410 would occur at differing portions of the lead body 410 at different times. This allows for the lead 400 to be remotely manipulated into complicated vascular structures. For example, the lead body can be manipulated into a device having a single curve or multiple curves in two or three dimensions.

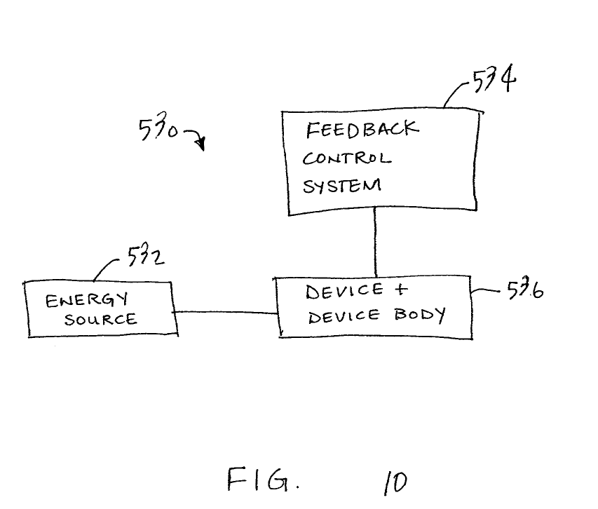

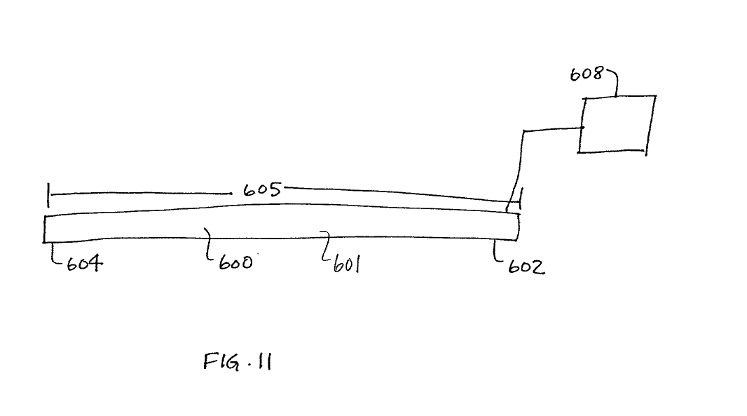

FIG. 7 illustrates yet another option for a medical device, such as a lead 500. The lead 500 includes a lead body 510 having a first surface 512 which is opposite a second surface 514. In one example, the lead 500 has a lead body 510 having a circular cross-section as shown in FIG. 8. In another example, the lead 500 has a lead body 510 having a square or rectangular cross-section as shown in FIG. 9. Referring again to FIG. 7, the lead body 510 extends to a distal end 502. One or more assemblies 520 are coupled with the lead body 510 on the first surface 512 and one or more assemblies 520 are coupled with the lead body 510 on the second surface 514. The one or more assemblies 520 are serially disposed along the lead body 510, and allow for the assemblies to be selectively activated. For example, a first assembly 560 is disposed along the lead body 510, a second assembly 562 is disposed adjacent to the first assembly 560, a third assembly 564 is disposed adjacent to the second assembly 562, and a fourth assembly 566 is disposed adjacent to the third assembly 564. Optionally, the fourth assembly 566 is disposed at or near the distal end 502 of the lead body 510. The one or more assemblies 520 also optionally comprise an electrode 523 and are adapted to provide and/or receive electrical signals to and from a heart. Each electrode 523 is electrically coupled with a conductor of the lead 500.

The one or more assemblies 520 include rheometric material associated therewith. Examples of rheometric material include, but are not limited to, magnoactive material or electroactive material such as an electroactive polymer. For instance, in one example, the one or more assemblies 520 each comprise a layer of polymer 522 with electrodes 524 deposited thereon. The layer of polymer 522 comprises an electrically active polymer. In one example, the layer of electrically active polymer 522 is a film of polymer about 180 micron thick. Other thicknesses of the layer of electrically active polymer 522 are suitable as well. The electrodes 524, which are electrically coupled with a conductor of the lead 500, comprise a metallic coating which is deposited on opposite surfaces 526, 528 of the layer of electrically active polymer 522. In one example, the metallic coating is comprised of platinum. The passive properties of the layer of electrically active polymer 522 and the metallic coating are modifiable, as well as the serial placement of the one or more assemblies 520, to alter the flexibility of the lead 500.

The electrodes 524 allow for a voltage to be applied across the layer of electrically active polymer 522. When voltage is applied to the electrodes, for example 2-7 volts, an electric field is established, which causes the layer of electrically active polymer 522 to contract. As the layer of electrically active polymer 522 contracts in a first direction, it also expands along “G,” causing the lead body 510 to bend toward the opposite side in a bending moment. However, an assembly disposed on the opposite side would prevent the lead body 510 from bending, when undergoing the same type of bending moment.

For example, as voltage is applied to the first assembly 560 on the first surface 512 of the lead body 510, the electrically active polymer 522 of the first assembly 560 expands and produces a bending moment to the lead body 510 such that the lead body 510 bends toward the second surface 514. As voltage is applied to an assembly disposed on the second surface 514, the electrically active polymer 522 of the assembly expands along and produces a bending moment to the lead body 510 such that the lead body 510 bends toward the first surface 512. Since the bending moments oppose each other, the lead body 510 is stiffened thereby.

One option is to selectively apply voltage to the assemblies 520 along an intermediate portion of the device body to achieve an inchworm effect, so that the lead 500 can be manipulated into complex passages, such as vascular structures. For example, voltage is selectively applied to the assemblies as described above to accurately manipulate the device body, or achieve peristalsis effect. Other uses for the device body is for moving drugs from a proximal end of the device body along a passage of the device body, and delivering the drugs along a portion of the body, for example, at the distal end of the device body. One advantage is that the drugs can be delivered at different rates using this technique. Alternatively, the device body can be selectively stiffened to move a fluid from a distal end of the device body to a proximal end of the device body. For example, a blood sample can be drawn along the device body by selectively stiffening the device body to move the blood along a passage of the device body to a proximal end of the device body. Beneficially, the blood sample can be drawn slowly, and without trauma to the sample site.

Another option is to stiffen the lead 500, using any of the techniques discussed above, to selectively stiffen the lead 500 to brace the lead 500 against moving or contracting tissue. As the device body is being moved, or before the device body is moved by, for example, contracting tissue or blood flow, the rheometric material is used to stiffen the device body and minimize and/or prevent the device body from being moved by the environment of the patient. Bracing the lead 500 would allow for a more stable positioning of the lead 500, for example at the distal end 502 of the lead 500.

As mentioned above and below, voltage and/or current is applied to the rheometric material, resulting in a stiffening of the device body in a variety of different manners. In one option, the voltage and/or current is applied via an energy source included with the device body, for example a pulse generator included with a lead, where the low frequency alternating current is applied from the pulse generator to the lead. Alternatively, an external energy source can be electrically coupled with the device body. In another option, as illustrated in FIG. 10, an assembly 530 includes a device having a device body 536, for example, any of the above and below described devices. The device body 536 is electrically coupled with an energy source 532. The energy source 532 is configured to apply voltage and/or current to the rheometric material of the device body 536, resulting in a stiffening of at least a portion of the device body 536.

In another option, the assembly 530 further includes a feedback control system 534. For instance, the device body 536 optionally includes a marker or other material which allows for movement or location of the device body 536 to be monitored, for example by an imaging system. One example of a marker is fluoroscopic material coupled with the device body 536. As the movement or location of the device body 536 is monitored and/or analyzed by the feedback control system 534, selective application of the voltage and/or current is conducted to manipulate the device body 536 in a prescribed movement, or to brace the device body 536 against an anticipated movement. In another option, other options for providing feedback are incorporated into the assembly 530. For instance, a pressure sensor is included with the assembly, providing information about the environment in which the device is placed. In another option, a strain gauge, a force sensing resistor, or an accelerometer is incorporated into the device. It should be noted that one or more of the options can be combined to achieve enhanced feedback, and to achieve more complex manipulation of the device body.

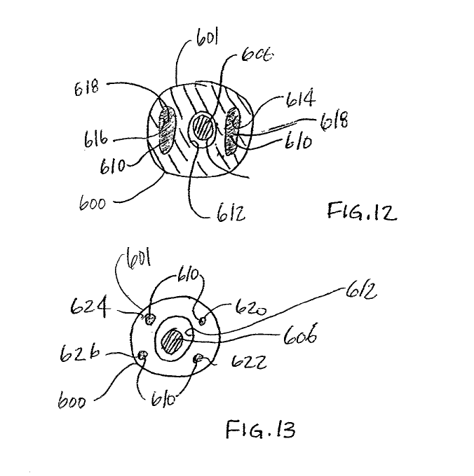

FIGS. 11-21 illustrate another medical device including rheometric material, for example, a guide catheter 600. However, it should be noted that other medical devices are suitable as well. For example, other suitable medical devices include, but are not limited to: medical device for chronic or acute use, catheter, lead, endoscope, ablation tool, pressure measuring tool, endoscope, or a blood sampling device. The medical device is suitable for use in combination with the above described embodiments, and is suitable for use with the above described methods. Examples of rheometric material include, but are not limited to, magnoactive material or electroactive material, such as an electroactive polymer, and the rheometric materials in above discussed embodiments.

Referring to FIG. 11, the guide catheter 600 extends from a proximal end 602 to a distal end 604, and is defined in part by a length 605. The guide catheter 600 is sized and/or configured to be manipulated and steered within tissue, for example, within vasculature of a body, and optionally has an elongate structure. The guide catheter 600 includes, in one option, tubular polymeric material which allows for instruments, such as implantable leads, therethrough.

As shown in FIGS. 12-19 and 21, the guide catheter 600 has a device body 601 that includes at least one passage 612 extending from a proximal end 602 (FIG. 11) to a distal end 604 (FIG. 11) of the guide catheter 600. The passage 612 is sized to receive at least one instrument 606 therein, for instance a lead. Other instruments are suitable as well. It should be noted that the instrument 606 is, in one option, integral with the guide catheter 600. In another option, the guide catheter 600 is movable relative to the instrument. For instance, the guide catheter 600 can be removed from a patient, while the instrument 606 remains therein. In yet another option, fluids can be moved through the passage 612. It should be noted that guide catheter 600 optionally includes one or more passages 612 therein.

Guide catheter 600 is particularly suited for moving through complex passages of a body, for instance, through the coronary sinus and into the ostium. In one option, the guide catheter 600 includes rheometric material associated therewith. The guide catheter 600 is electrically coupled with an energy source 608 (FIG. 11), for example, an external energy source, where the energy source 608 (FIG. 11) is electrically coupled with the rheometric material. When electric current is applied to the rheometric material, the rheometric material causes the device body to stiffen, for instance the rheometric material stiffens. In one option, the electrically activated material includes the materials discussed above, including, but not limited to, electrically active polymers. In another option, the electrically activated material includes electroactive materials or magnoactive materials, and the materials of the earlier discussed embodiments.

In one option, the guide catheter 600 includes at least one lumen 610 therein, and at least one lumen 610 has rheometric material disposed therein. In another option, the rheometric material is associated with the guide catheter 600 as in the above discussed embodiments. In yet another option, the rheometric material is disposed within a plurality of lumens 610. It should be noted that the cross-sectional shape, geometry, number, length, and configurations of the lumens which receive the rheometric material therein are modifiable in several configurations, as shown by way of example, in FIGS. 11-21.

For instance, in one option shown in FIGS. 12 and 13, the at least one lumen 610 includes a first lumen 614 and a second lumen 616 which are disposed on opposite sides of the passage 612. In one option, the at least one lumen 610 or the first lumen and the second lumen 616 extend from the distal end 604 (FIG. 11) to the proximal end 602 (FIG. 11) of the guide catheter 600. Disposed within the first lumen 614 and the second lumen 616 is a rheometric material 618, such as a magnoactive material or an electroactive material. In another option, as shown in FIG. 13, the at least one lumen 610 includes a plurality of lumens 620, 622, 624, and 626, where each lumen 620, 622, 624, and 626 includes rheometric material disposed therein.

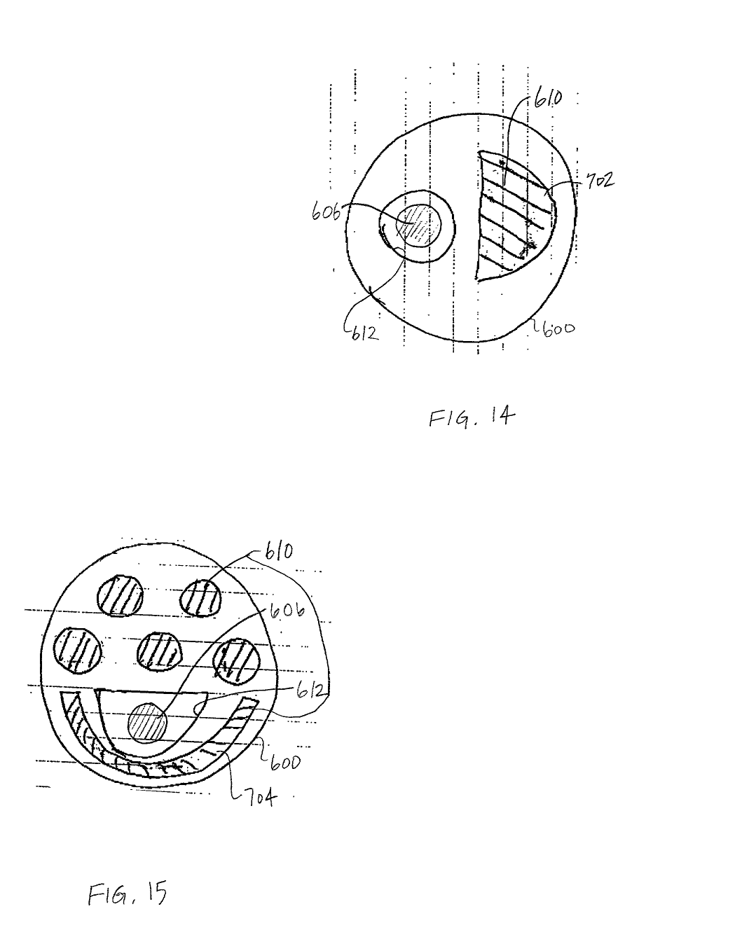

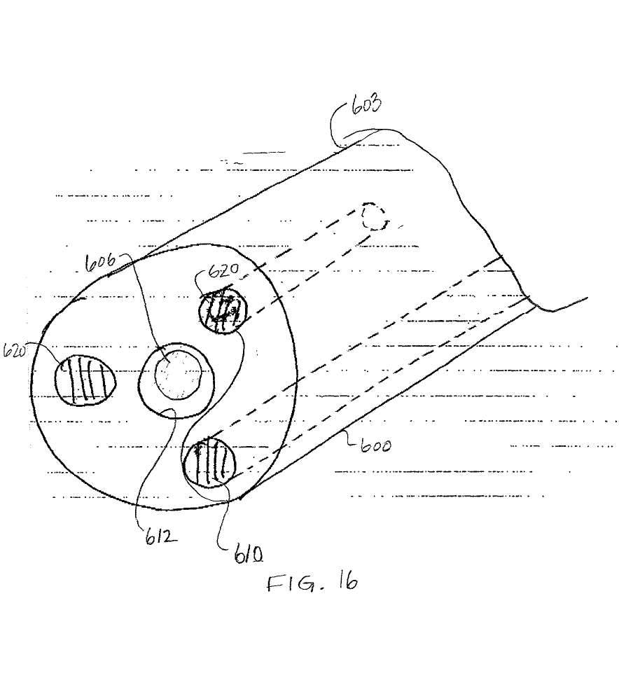

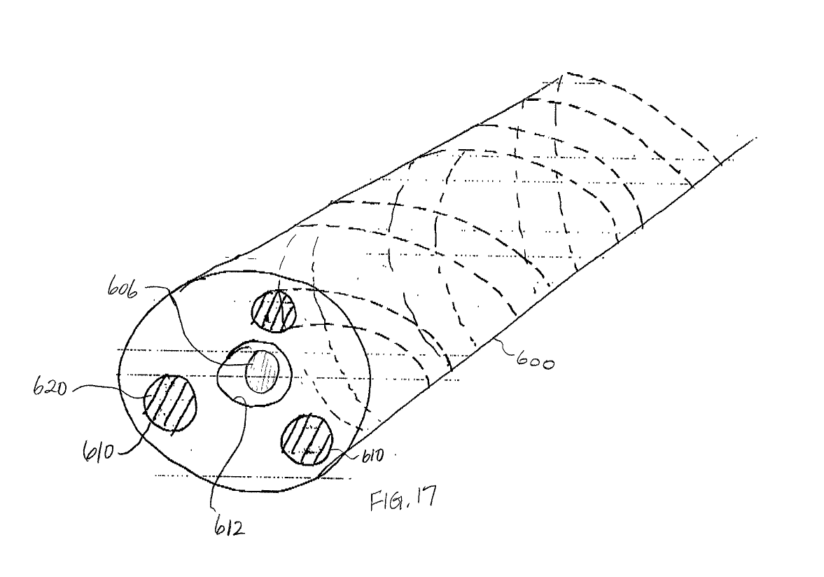

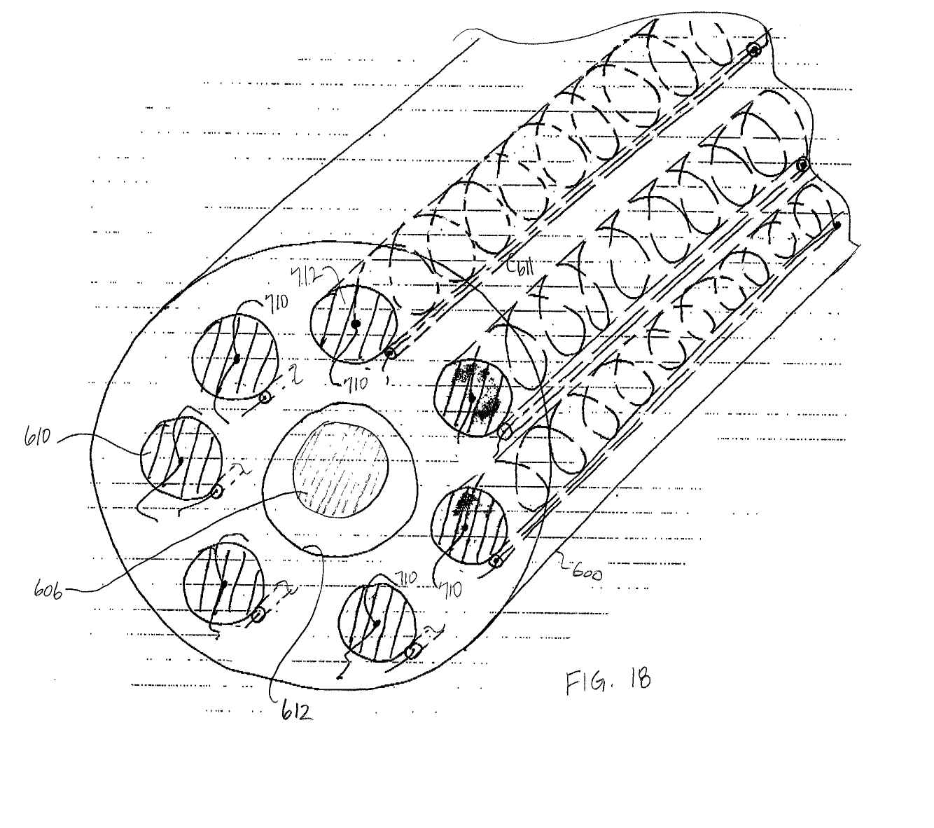

In another embodiment, as shown in FIG. 14, the lumen 610 which receives rheometric material therein has a semi-circular cross-section 702. Another option, as illustrated in FIG. 15, the at least one lumen 610 has a C-shape 704 which at least partially surrounds the passage 612 which receives the instrument therein. FIG. 16 illustrates yet another option for the medical device. The plurality of lumens 620 are equally spaced about the passage 612. The lumens 620 extend along a longitudinal axis of the medical device. At least one of the lumens 620 extends for only a portion of the length of the medical device, and stops at an intermediate portion 603 of the device body. Discontinuous lumen lengths provides differential stiffening along the length of the device body when applying energy to the various lumens. FIG. 17 illustrates a plurality of lumens 620 disposed about passage 612. One or more of the lumens 620 is a swirled lumen 621 which wraps about the longitudinal axis of the catheter 600. The swirled lumens form a helical shape around the longitudinal axis of the catheter 600, and allow for torque to be applied to the device body as energy is applied to the rheometric material.

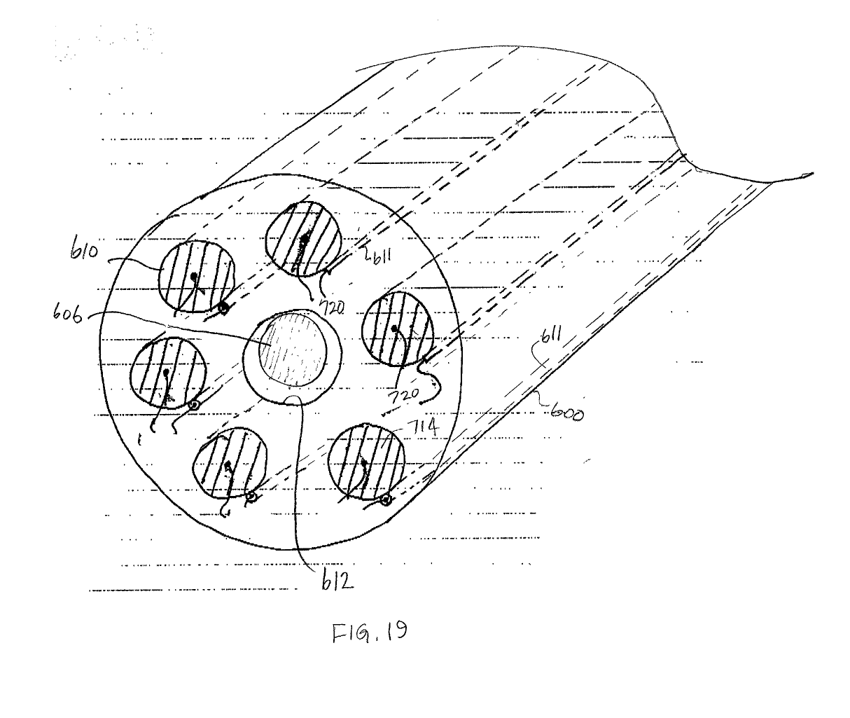

During operation of the medical device, energy is applied to the rheometric material. FIGS. 18 and 19 illustrate one example of the application of energy. In one option, as shown in FIG. 18, at least one conductor 710 is disposed in each lumen 610, and returns in a secondary lumen 611. The conductor 710, is coiled within the lumen 610, and is coupled with an energy source 608 (FIG. 11). Applying energy to the at least one conductor 710 creates a magnetic field within the lumen 610 and electrically activates the magnoactive material 712 therein.

In another option, as shown in FIG. 19, at least one conductor 720 is disposed in each lumen 610, and returns in a secondary lumen 611. The conductor 710 is coupled with an energy source 608 (FIG. 11). Applying energy to the at least one conductor 720 electrically activates the rheometric material, such as the electroactive material 714 therein.

As energy is applied to the rheometric material from the energy source 608 (FIG. 11), the guide catheter 600 is stiffened along its longitudinal axis. By varying current to the rheometric material of one or multiple lumens, the guide catheter would be pulled or pushed along the sides of the catheter 600. As the catheter 600 is pushed and/or pulled on its side, the catheter 600 moves from side to side, allowing for steering of the distal end of the catheter 600. Alternatively, the rheometric material can be distributed along the device in different manners, as discussed above and below. The energy can be selectively applied to achieve more complex manipulations. For example, the distal end 604 (FIG. 11) of the guide catheter can be steered into the right coronary sinus ostium.

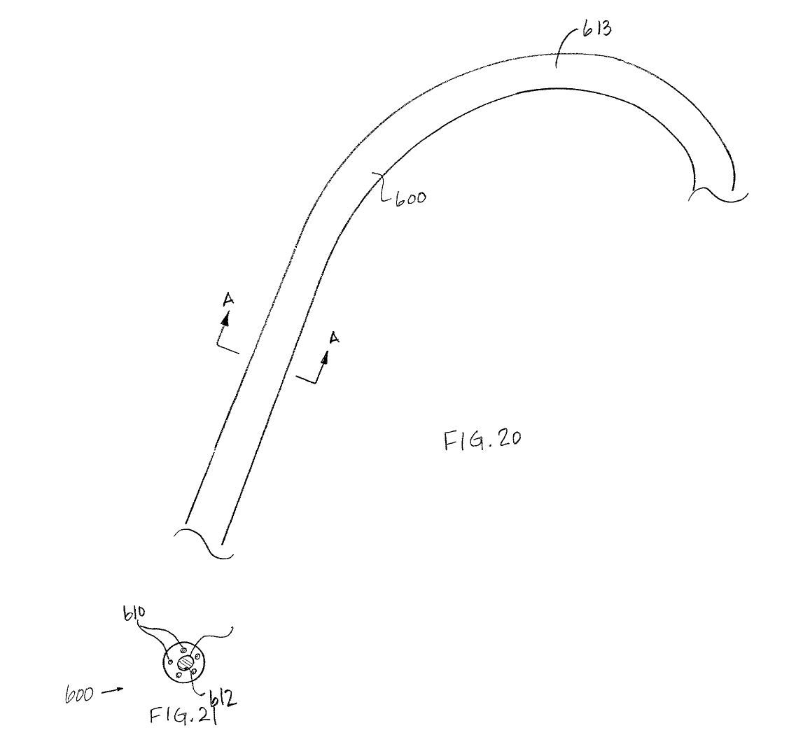

FIGS. 20 and 21 illustrate yet another embodiment of a medical device including rheometric material associated therewith. The guide catheter 600 includes, in one option, a device body 601 that is preformed with a curve 613 therein. The body is formed with the curve 613 during, for example, the manufacturing process, and not during the implantation process. The electro-rheological or magneto-rheological material is introduced into the lumens after the device body is formed. In one option, as electrical energy is applied to the device, the device body is straightened.

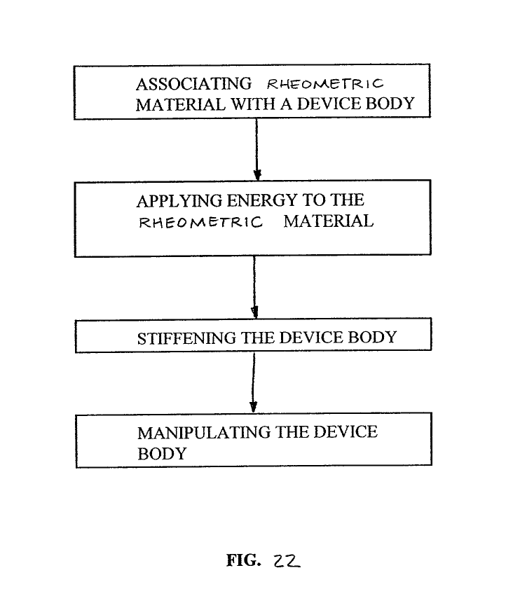

Referring to FIG. 22, a method for manipulating a medical device is described herein. It should be noted that the method includes the above and below discussed device embodiments described herein. Although some of the embodiments are discussed in the context of a lead or a catheter, the method applies to a wide variety of medical devices, including, but not limited to, medical devices for chronic or acute use, catheters, leads, endoscopes, ablation tools, pressure measuring tools, or blood sampling devices.

The method includes associating rheometric material with a device body, such as an elongate device body. For instance, the method includes associating at least one assembly with the device body, where the at least one assembly optionally includes at least one electrode. The method further includes applying energy to the rheometric material, stiffening at least a portion of the device body, and manipulating the device body.

Several options for the method are as follows. For instance, in one option, applying energy to the assembly comprises applying voltage to multiple assemblies each including at least one electrode electrically coupled with a layer of electroactive polymer. The energy is optionally applied to each assembly simultaneously, or selectively applied energy to each assembly at different times. Still further, in another option, applying energy includes applying voltage to an assembly which is wound around an axis of the device body, or to an assembly disposed at a distal end of the device body, or to a plurality of assemblies disposed on a single side of the device body, or to a plurality of assemblies disposed on at least two sides of the device body. Optionally, the assemblies are disposed within the device body or are disposed on one or more outer surfaces of the device body.

In other options for the method, the method further includes selectively varying stiffness of the device body, where selectively varying the stiffness of the device body includes moving the device body within a passage, or bracing the device body against movement, or moving fluid through the device body.

In another embodiment, a method includes providing an elongate device body having a length, associating a rheometric material along at least a portion of the length, applying an electric current to the rheometric material, and stiffening at least a first portion of the device body.

Several options for the method are as follows. For instance, in one option, applying electric current includes pulsing the electric current and alternately stiffening and relaxing the first portion of the device body. Alternatively, stiffening includes stiffening the entire length of the device body. In yet another option, the device body includes one or more lumens therein, and associating includes disposing rheometric material in at least one lumen of the device body, or in at least two or more lumens of the device body. In yet another option, applying electric current includes pulsing the electric current and alternately stiffening and relaxing multiple portions of the device body. In yet another option, the device body is preformed with a curve. The body is formed with the curve during, for example, the manufacturing process. In one option, as electrical energy is applied to the device, the device body is straightened.

Advantageously, movement of the above-described medical device is controllable from an outside source, without having to implement invasive procedures, or without having to rely exclusively on additional instruments such as stylets. In addition, the stiffness of the medical device can also be modified at different portions and at different time periods which allows for the resistance of movement of the device in response to, for example, a beating heart. Alternatively, the ability to selectively and independently modify the stiffness of the lead along different segments, at different times allows for the position of the medical device to be manipulated within the patient, without further invasive procedures, and allows for the device to be manipulated into complex configurations, such as within the human vasculature. Another provided benefit is that the device can be braced against moving tissue, and/or for procedures in which the device moves during the procedure.

Furthermore, since the medical device can be manipulated into more precise locations, under more demanding conditions, improved positioning of the device can be achieved, resulting in improved performance of the medical device. For example, delivering energy to a more favorable location on the heart results in a better chance for a more-effective defibrillation.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

What is claimed is:

1. A medical device comprising:
a device body extending from a proximal end to a distal end;
at least one electrode coupled with the device body, where the at least one electrode is configured to transmit and receive electrical signals to and from tissue; and
a rheometric material electrically coupled with the at least one electrode.

2. The medical device as recited in claim 1, wherein the rheometric material comprises a coating of electroactive polymer having a thickness of about 180 micron.

3. The medical device as recited in claim 1, wherein the rheometric material comprises a strip of material wound around a longitudinal axis of the device body.

4. The medical device as recited in claim 1, wherein the rheometric material comprises a layer of material on an outer surface of the at least one electrode.

5. The medical device as recited in claim 1, wherein the device body is defined by a first surface and a second surface, and the at least one electrode is disposed on the first surface of the device body.

6. The medical device as recited in claim 5, wherein the first surface is opposite the second surface, and at least one electrode is disposed on the second surface of the device body.

7. The medical device as recited in claim 1, wherein the device body comprises an elongate lead body configured to be coupled with a pulse generator.

8. The medical device as recited in claim 1, wherein the rheometric material comprises an electroactive polymer.

9. A medical device comprising:
an elongate device body extending from a proximal end to a distal end;
at least one assembly coupled with the device body, where the at least one assembly is configured to stiffen the device body; and
the at least one assembly including a rheometric material, the rheometric material contracts and/or stiffens when electrical current is applied thereto.

10. The medical device as recited in claim 9, further comprising a control system which selectively applies current to the rheometric material, and a means for providing feedback to the control system.

11. The medical device as recited in claim 9, further comprising a means for transferring fluid along the elongate device body.

12. The medical device as recited in claim 9, wherein the device body is defined by a first outer surface and a second outer surface, and the at least one assembly is disposed on the first outer surface of the device body.

13. The medical device as recited in claim 12, wherein the first outer surface is opposite the second outer surface.

14. The medical device as recited in claim 9, wherein a plurality of assemblies are disposed on a first outer surface of the device body.

15. The medical device as recited in claim 9, wherein the device body includes a first outer surface and a second outer surface, and a plurality of assemblies are disposed on the first outer surface, and a plurality of assemblies are disposed on the second outer surface.

16. The medical device as recited in claim 9, wherein the at least one assembly is disposed adjacent to the distal end of the device body.

17. The medical device as recited in claim 9, wherein the assembly is disposed within at least one lumen of the device body along at least a portion of a length of the device body.

18. The medical device as recited in claim 17, wherein at least one assembly is disposed along the entire length of the device body.

19. The medical device as recited in claim 17, wherein the device body includes two or more lumens therein, and at least one lumen has a different cross-section than another lumen, and rheometric material is disposed within the two or more lumens.

20. The medical device as recited in claim 9, wherein the rheometric material comprises magnoactive material.

21. The medical device as recited in claim 9, wherein the rheometric material comprises electroactive material.

22. The medical device as recited in claim 9, wherein the device body has a preformed curved portion.

23. A medical device comprising:
a device body extending from a proximal end to a distal end;
at least one assembly coupled with the device body, the at least one assembly includes at least one winding of material wound around a longitudinal axis of the device body, where the at least one assembly is configured to stiffen the device body; and
the at least one assembly including a rheometric material, the rheometric material contracts and/or stiffens when current is applied thereto.

24. The medical device as recited in claim 23, wherein the rheometric material is an electroactive polymer coating of about 180 micron in thickness.

25. The medical device as recited in claim 23, wherein the winding of material extends from the proximal end to the distal end of the device body.

26. The medical device as recited in claim 23, further comprising a control system which selectively applies current to the electroactive material, and a means for providing feedback to the control system.

27. The medical device as recited in claim 23, wherein the winding of material is disposed within one or more lumens of the device body.

28. A medical device comprising:
an elongate device body extending from a proximal end to a distal end;
at least one assembly coupled with the device body; and
means for electrically stiffening the at least one assembly and the device body.

29. The medical device as recited in claim 28, wherein the at least one assembly includes an electroactive polymer associated therewith.

30. The medical device as recited in claim 28, wherein the at least one assembly includes magnoactive material associated therewith.

31. The medical device as recited in claim 28, wherein the device body includes at least one lumen therein, and rheometric material is disposed within one or more lumens.

32. The medical device as recited in claim 31, wherein the device body further includes at least one lumen configured to receive a medical instrument or fluid therethrough.

33. The medical device as recited in claim 28, wherein the device body has a preformed curve.

34. A medical device comprising:
an elongate device body extending from a proximal end to a distal end;
the device body including at least one lumen therein, and rheometric material is disposed within one or more lumens, the rheometric material configured to stiffen the elongate device body upon application of electrical energy to the rheometric material.

35. The medical device as recited in claim 34, wherein the rheometric material includes an electroactive polymer.

36. The medical device as recited in claim 34, wherein the rheometric material includes magnoactive material.

37. The medical device as recited in claim 34, wherein the device body includes a passage extending from the proximal end to the distal end, the passage sized to receive at least one instrument therein, and a plurality of lumens are disposed about the passage.

38. A method comprising:
associating at least one assembly with a device body, the at least one assembly including at least one electrode;
electrically coupling a rheometric material with the at least one electrode;
applying energy to at least one assembly; and
the rheometric material stiffening at least a portion of the device body.

39. The method as recited in claim 38, wherein applying energy comprises applying voltage to multiple assemblies each including at least one electrode electrically coupled with a layer of electroactive polymer.

40. The method as recited in claim 38, wherein applying energy includes applying energy to each assembly simultaneously.

41. The method as recited in claim 38, wherein applying energy includes selectively applying energy to each assembly at different times.

42. The method as recited in claim 38, wherein applying energy includes applying voltage to an assembly which is wound around an axis of the device body.

43. The method as recited in claim 38, wherein applying energy includes applying energy to an assembly disposed at a distal end of the device body.

44. The method as recited in claim 38, wherein applying energy includes applying voltage to a plurality of assemblies disposed on a single side of the device body.

45. The method as recited in claim 38, wherein applying energy includes applying voltage to a plurality of assemblies disposed on at least two sides of the device body.

46. The method as recited in claim 38, further comprising selectively varying stiffness of the device body.

47. The method as recited in claim 46, wherein selectively varying the stiffness of the device body includes moving the device body within a passage.

48. The method as recited in claim 46, wherein selectively varying the stiffness of the device body includes bracing the device body against movement.

49. The method as recited in claim 46, wherein selectively varying the stiffness of the device body includes moving fluid through the device body.

50. A method comprising:
providing an elongate device body having a length;
associating rheometric material along at least a portion of the length;
applying an electric current to the rheometric material; and
stiffening at least a first portion of the device body.

51. The method as recited in claim 50, wherein applying electric current includes pulsing the electric current and alternately stiffening and relaxing the first portion of the device body.

52. The method as recited in claim 50, wherein stiffening includes stiffening the entire length of the device body.

53. The method as recited in claim 50, wherein the device body includes one or more lumens therein, associating includes disposing rheometric material in at least one lumen of the device body.

54. The method as recited in claim 50, wherein the device body includes one or more lumens disposed along at least a portion of longitudinal axis of the device body, and wherein associating material includes disposing rheometric material in two or more of the lumens.

55. The method as recited in claim 50, further comprising stiffening multiple portions of the device body.

56. The method as recited in claim 50, wherein applying electric current includes pulsing the electric current and alternately stiffening and relaxing the multiple portions of the device body.

57. The method as recited in claim 50, further comprising preforming the elongate device body with a curve.
